(19) United States
(12) Patent Application Publication
(10) Pub. No.: US 2017/0266409 A9
(43) Pub. Date: Sep. 21, 2017

(54) Process and System of Matching Harvesting and Reinjection Cannulas for Use in Removal and Reinjection Procedures of Adipose Tissue, Other Tissues, and Fluids Commonly Called Liposuction

(71) Applicant: John F. Wells, Tucson, AZ (US)

(72) Inventor: John F. Wells, Tucson, AZ (US)

(73) Assignee: Wells Johnson Company, Tucson, AZ (US)

(21) Appl. No.: 14/742,496

(22) Filed: Jun. 17, 2015

Related U.S. Application Data
(60) Provisional application No. 62/179,885, filed on May 22, 2015.
(60) Provisional application No. 62/178,756, filed on Apr. 20, 2015.
(60) Provisional application No. 62/177,503, filed on Mar. 17, 2015.

(51) Int. Cl. A61M 25/00

(52) U.S. Cl. CPC A61M 25/007; A61M 25/002; A61M 2202/08

(57) ABSTRACT

A process and system of compatible cannulas with features that are linked and intentionally related including the Interior Cross Sectional Area (302) of the cannulas, the area of the opening(s) of the cannulas and the width of the opening(s) of the cannulas (303). When used in a method where the appropriate harvesting cannula (100) is selected for use with a desired reinjection cannula (100), the cannula kit (FIG. 8) provides increased benefit for maintaining live tissue viability, and minimizes the time of the surgical procedure. The cannula (100) as a kit or set (FIG. 8) is used for harvesting and for reinjection, and serves in extracting, processing and transferring adipose tissue.

### CROSS REFERENCE TO RELATED PATENT APPLICATION(S)

This application is a non-provisional application being filed under 37 CFR 1.53(b) and 35 USC 111 as a divisional of the presently pending U.S. patent(s) all of which are hereby incorporated by reference.
Application No. 62/177,503
Filing Date—Mar. 17, 2015
Title—Compatible set of harvesting and reinjection cannulas
Application No. 62/178,756
Filing Date—Apr. 20, 2015
Title—Method and control of pressure when handling adipose (related)
Application No. 62/179,885
Filing Date—May 22, 201
Title—Process and System for Fluid Management During Reinjection of Adipose Tissue

### BACKGROUND AND SUMMARY OF THE INVENTION

The present invention relates generally to the field of invasive surgical instruments used during cosmetic surgery and reconstruction of the breast after breast cancer for strengthening breast tissue. More specifically, the present invention relates to an improved liposuction kit for removing excess subcutaneous fat from the body and replacing by reinjecting it into the same body during said procedure. The invention is a kit that includes both parts, a harvesting cannula and a reinjection cannula with the size of the holes being intentionally matched to be substantially equal.

Body fat comes in various different forms and structures and varies in composition in different regions of the body. Much subcutaneous fat in a human often forms a thin blanket-like layer. In some areas of the body fat cells exist in large subcutaneous deposits. Adipose tissue consists of fat cells and fibrous tissue. Fat cells store lipids. A large variety of metabolic disorders or dietary habits can cause fat deposits to become excessively enlarged, with undesirable cosmetic and medical side effects.

Liposuction is an invasive surgical procedure where subcutaneous fat cells are removed by the use of a liposuction cannula attached to a vacuum suction device. The cannula is percutaneously inserted below the derma skin layer into subcutaneous deposits of fat through very small (4 mm to 10 mm) incisions. As the cannula is carefully moved to and fro, fat cells are dislodged by the force of the applied vacuum and simultaneously suctioned away via a long flexible hose connecting the cannula to the vacuum pump collection bottle.

Until recently, the removed adipose tissue was only discarded. In recent applications, it has been reinjected into the body to be placed in a more desirable location sometimes being needed to improve the reconstruction of breasts after breast cancer which can thicken the skin or outer dermal wall of the patients' breast tissue.

This technique has been used to reform the shape of different parts of the body not only for aesthetic purposes, but for reconstruction and health related purposes. A kit of matched harvesting and reinjection cannulas has never been introduced or offered in the market previously.

The matching of any of the features of the harvesting and reinjecting cannulas has never been cited or previously researched as a significant characteristic of a kit sold as a matching set for harvesting and reinjection in the same procedure. The reinjection cannula has been chosen for the purposes of the operator's preferences, it's handle size and probe size to match the reinjection opening produced in the patient's body.

As a kit, the invention includes matching the sizes of the side holes in the cannula where fat cells enter into the harvesting cannula and where they exit out of the reinjection cannula.

The invention refers to openings at the distal end, which refers to machined holes on the side of the cannula lumen, not an opening at the distal end. Generally, the cannula has a rounded, closed tip at the distal end. The machined holes referred to in the claims and the descriptions within the specifications are specifically machined into the side of the cannula lumen with the end sealed or closed.

The use of the kit is intended to be utilized during the procedure used to reform the shape of different parts of the body not only for aesthetic purposes, but for reconstruction and health related purposes.

The present invention pertains to a kit with intentionally matched properties, and the actual physical system of linked features of harvesting and reinjection cannulas used for adipose tissue removal and reinjection during liposuction. In the present art, there exists a large number of variations in the size, length and hole pattern of these cannulas and this invention focuses on a kit that matches at least one of the three major features in the preferred embodiment, but implies there are and could be many more that are later discussed. The three major features include:

I. Inner cross sectional area of the lumen

II. The area of an opening at or near the distal end and on a side of a cannula

III. The width of an opening at or near the distal end and on a side of a cannula

Harvesting cannula openings tend to be the most restrictive of all passages in the transfer system. The adipose tissue needs to exit the openings of the reinjection cannula with minimal resistance. Linking the features described above, or any combination of them, is a form of processing of the adipose tissue. Rather than take in any size agglomerate and try to separate and process them later, the entire processing step takes place before the agglomerates enter the system. This ensures that the size and shape of agglomerates that enter the inner lumen area of the harvesting cannula will be able to pass through and exit the reinjection cannula.

While filtering and processing the adipose tissue is necessary, the speed with which the liposuction procedure is completed is another important factor. Flow rates directly impact the speed with which adipose tissue is drawn into the harvesting cannula. The process of matching characteristics between the harvesting and reinjection cannulas, such as an inner cross sectional area and the dimensions of an opening, ensures maximum transfer of undamaged tissue is achieved. This allows transfer of cells from one part of the body to another at a high speed with minimal damage or trauma to the cells.

### GENERAL DESCRIPTION OF THE VIEWS OF THE DRAWINGS

FIG. 1 is a Block Diagram of the process of selecting and matching the cannula characteristics of the kit, represented in several steps 121-126. These steps are in a chronological order of how the kit is used.

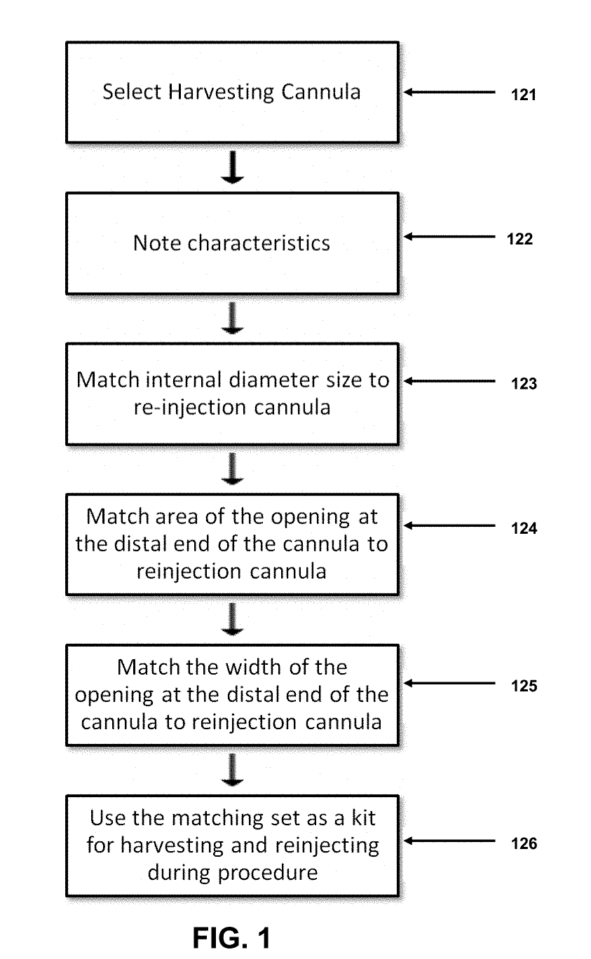

FIG. 2 is a Flowchart Illustration of an embodiment of the invention which represents choices made by the user of the kit to remove the fat and replace it with the most potential success and viability of cells that are disturbed from transport of one location to a new location within the human body. These choices include the correct reinjecting cannula and then intentionally choosing the correct, and matching, harvesting cannula.

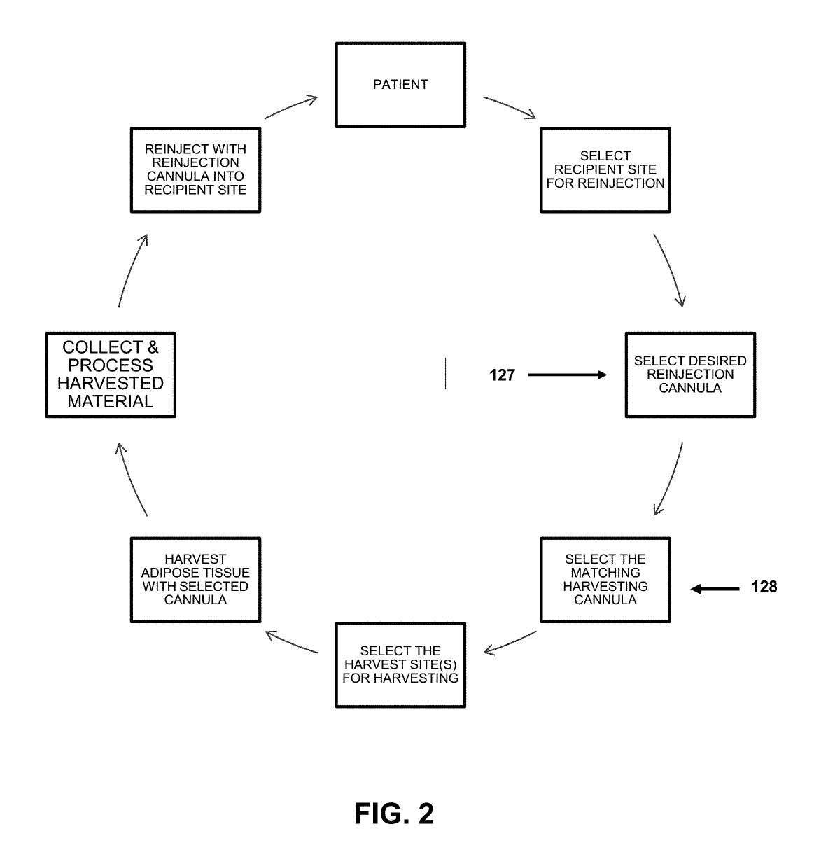

FIG. 3 is a Block Diagram of the actual path of the fat and other materials from initial harvesting to reinjection back into the patient. Each step is detailed 130-137.

FIG. 4 is a Schematic View of one embodiment of the invention.

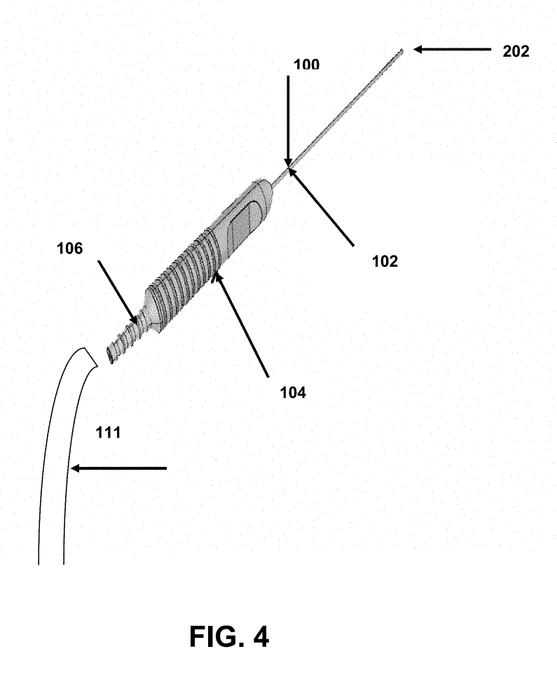

FIG. 5 is a Rear View of one embodiment of the invention.

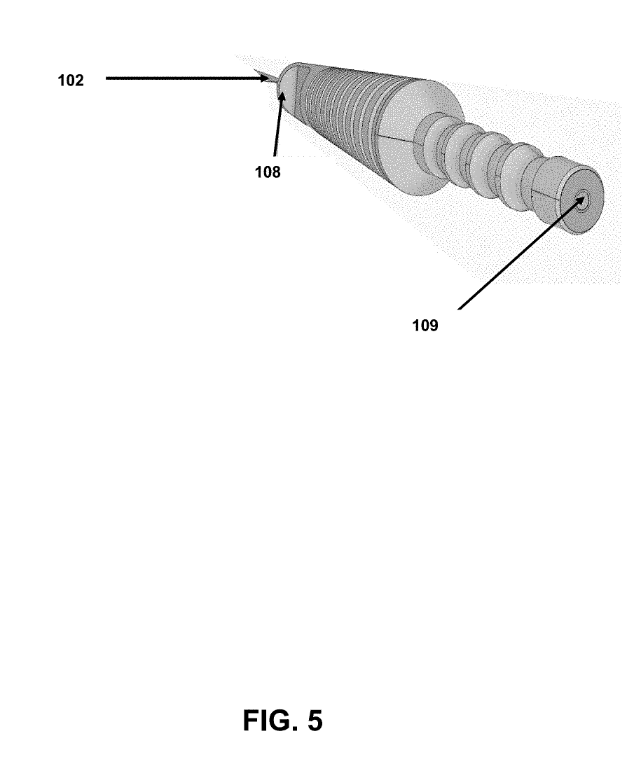

FIG. 6 is a Full Schematic of a typical cannula embodied by the invention.

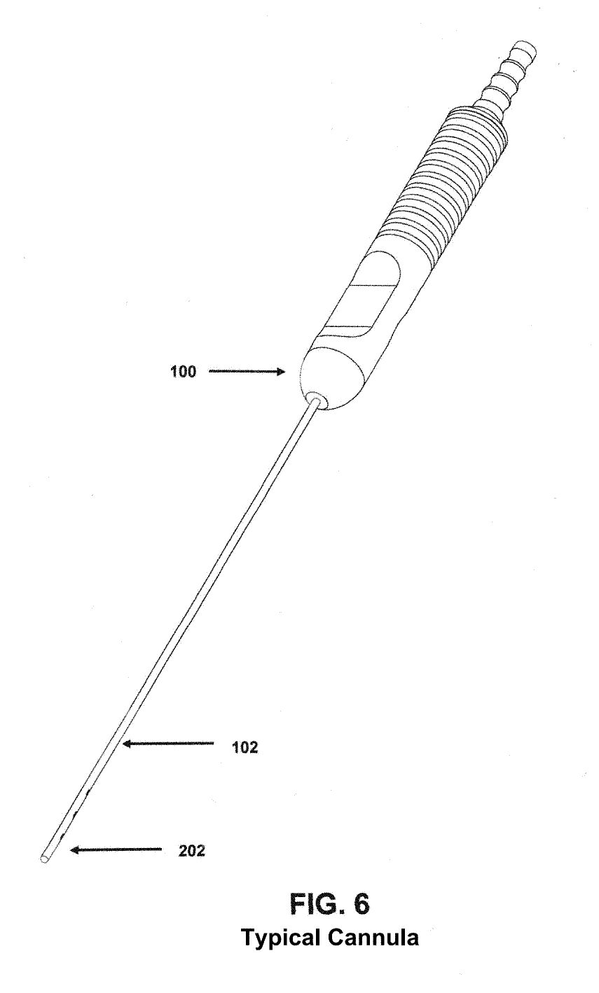

FIG. 7 contains a transparent side view of the distal end of a cannula with one possible example of opening sizes and inner cross sectional areas.

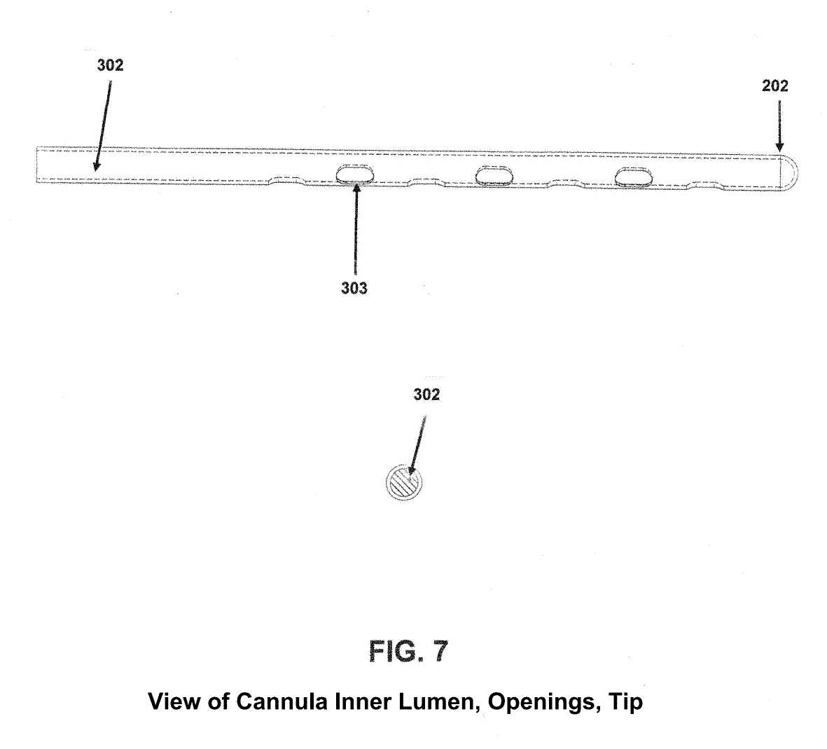

FIG. 8 is a cannula kit with one possible example of matching harvesting and reinjection cannulas with matching opening sizes and inner cross sectional areas.

FIG. 9 is a transparent side view of the distal end of a cannula with one possible example of an array of openings of some size and shape.

### DETAILED DESCRIPTION OF DRAWINGS AND EMBODIMENTS OF THE INVENTION

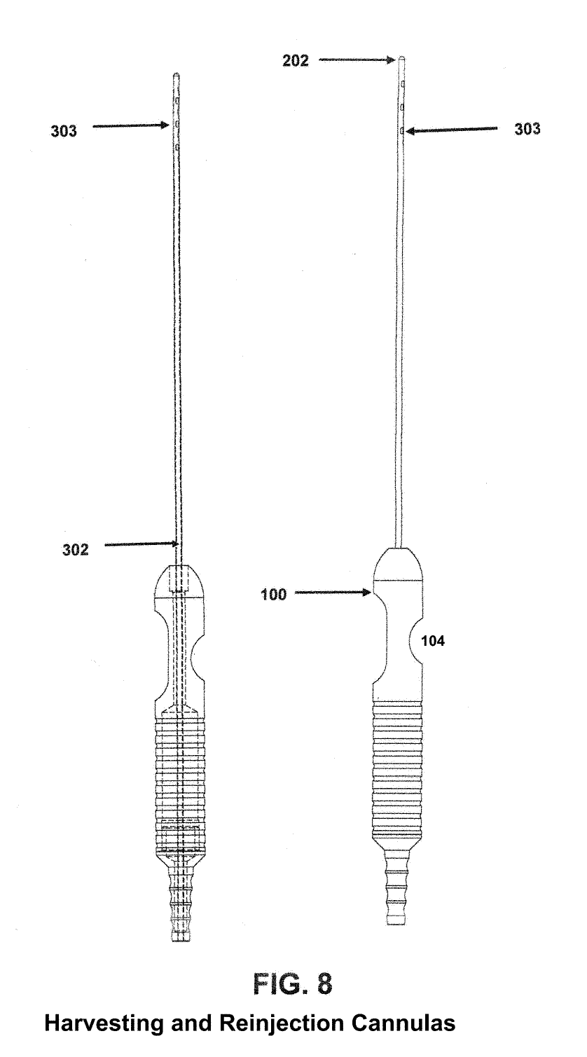

The cannulas 100 are used for harvesting and reinjection of adipose tissue. (FIG. 8) Upon harvesting, the harvesting cannula 100 is used within the body and moved around as the vacuum pressure causes a suction at the openings 303 in the side of the cannula 100.

The term adipose is known to those skilled in the art, and one non-limiting definition, provided for convenience only, includes fatty skin tissue, or skin tissue consisting of, resembling, or relating to fat. Adipose tissue is a type of connective tissue consisting of adipose cells, which are specialized to produce and store large fat globules.

The term “cannula” 100 as used in the text of the present disclosure, means any flexible or rigid light guide 102 configured to be inserted into the subject tissue to deliver or remove adipose tissue. In the preferred embodiment, there is generally an internal hole or open tube within the probe running the length of the cannula 100 representing a continuous internal passage way. One end of the cannula 100 may contain a barbed end 106 where the silicone or rubber tubing 111 is attached, and a handle 104 grip. The barbed end 106 having an exit 109 for delivery of the adipose tissue. One embodiment of the handle 104 would include a thumb indentation 108 for easy gripping and handling.

The distal end 202 refers to the end of the cannula 100 that is inserted in the body and is opposite of the handle 104 which contains the barbed end 106 onto who which rubber, plastic or silicon tubing 111 may be attached to provide suction or positive pressure.

The tip of the distal end 202 of the cannula 100 is generally a rounded, machined end that is closed off from suction and is smooth to the touch so that it causes no destruction and moves freely in the body. This kind of tip is generally referred to as a blunt tip. The tip is inserted carefully by the surgeon, into the body where either fat harvesting or reinjection will be performed.

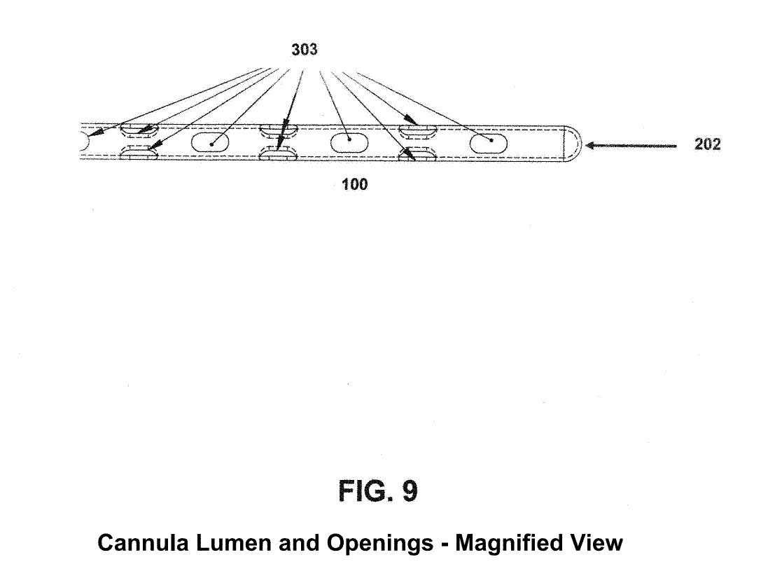

The harvesting openings 303 can vary in size and number and are not limited in size or number. The openings 303 are holes that are cut or machined into the side of the cannula 100 which appears to be a hollow tube with the tip closed. No harvesting or reinjection openings 303 are on the very tip or at the distal end 202 of the cannula 100 and the distal end 202 is not open but completely closed so as to avoid harvesting fat cells through the tip. This is a distinct difference to a needle, which uses a sharp, open tip. Often times, the side openings 303 are oval in shape and under a square centimeter but are not limited to a size restriction or a specific shape. The invention claims any shape or size of side openings 303. There is generally more than one opening 303 for the harvesting process. There is typically one opening 303 in the side of the cannula 100 for reinjection, however multiple openings 303 may be used as well, as seen in (FIG. 9). In the kit, the sizes of these openings 303 on the side are substantially similar in size between the harvesting and reinjection cannulas 100.

The current invention encompasses a “Kit” whereby a set of matching harvesting and reinjection cannulas 100 (FIG. 8), both of which have the side openings 303 used for harvesting and reinjection of fat cells during removal and reinjection procedures with intentional matching and compatible features are used and intended to maximize tissue viability and minimize procedure time when used in the removal and replacement of adipose tissue, said set comprising features including the following:
at least one blunt tip, closed end reinjection cannula 100 having a continuous, hollow tube provided with one or more reinjection openings 303 at or near a distal end 202 and on a side of the cannula 100, for reinjecting adipose tissue from an inner lumen of said hollow tube;
at least one blunt tip, closed end harvesting cannula 100 having a continuous, hollow tube provided with one or more harvesting openings 303 at or near a distal end 202 and on a side of the cannula 100, for drawing in adipose tissue into an inner lumen of said hollow tube;
where an area of one or more reinjection cannula 100 openings 303 on a side is ≧80% of an area of a harvesting cannula 100 opening 303 on a side.
where a width of one or more reinjection cannula 100 openings 303 on a side is ≧80% of a width of one or more harvesting cannula 100 openings 303 on a side, where a width of one or more reinjection cannula 100 openings 303 on a side is defined as the smallest dimension of one or more reinjection cannula 100 openings 303 on a side, and the width of one or more harvesting cannula 100 openings 303 on a side is defined as the smallest dimension of one or more harvesting cannula 100 openings 303 on a side.
where an inner cross sectional area 302 of a reinjection cannula 100 inner lumen is ≧30% of an area of one or more harvesting cannula 100 openings 303 on a side.

A kit of matching harvesting and reinjecting cannulas 100 for the movement of adipose tissue wherein the cannula 100 set is selected such that any of the preceding features of the reinjection cannula 100 used for a reinjection procedure match the features of the harvesting cannula 100 used for harvesting the adipose tissue for said reinjection procedure.

The principles and execution of the device, apparatus, and method described herein may be understood with reference to the drawings, wherein like reference numerals denote like elements through the several views and the accompanying description of non-limiting, exemplary embodiments.

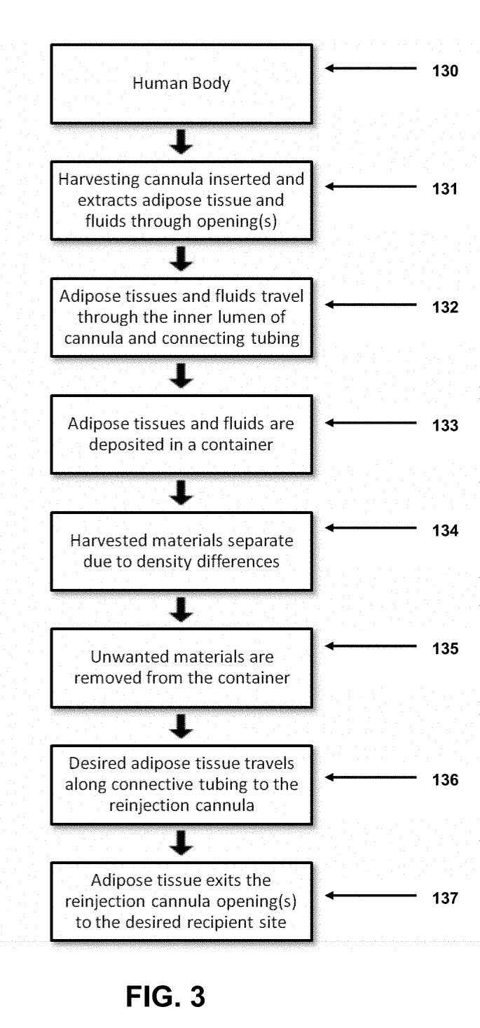

By way of example, and referring to FIG. 3, one embodiment of the invention pertains to the kit of cannulas 100 matching and acting together as a team where the passage way for the adipose tissue agglomerates moves them from the human patient 130 back to the human patient 130-137.

A harvesting cannula 100 opening 303 is intended to be the most restrictive of all passages in the transfer system. This means that a harvesting cannula 100 is processing the adipose tissue as it passes through a harvesting cannula 100 opening 303 simply because it is only allowing agglomerates of a limited size to enter the inner lumen of the cannula 100. It is found that these correctly sized agglomerates will more easily exit a reinjection cannula 100 when a compatible harvesting cannula 100 is selected.

In the kit, the general factors affecting compatibility include:
I. Inner cross sectional area 302 of a cannula 100 inner lumen
II. The area of an opening 303 at or near the distal end 202 and on a side of a cannula 100
III. The width of an opening 303 at or near the distal end 202 and on a side of a cannula 100

These factors ensure that any agglomerate that is successfully harvested through a harvesting cannula 100 opening 303 will easily exit a reinjection cannula 100 opening 303, regardless of orientation. These constraints allow a large variety of opening 303 sizes and orientations, while maintaining tissue viability and procedural efficiency.

Minimizing the time it takes to do each procedure is preferred in the process. It has been found that harvesting the adipose tissue takes a great deal of time and effort during a procedure. It is therefore important to apply some constraints to a harvesting cannula 100. A harvesting cannula 100 in the described set will have the following:
1. A multitude of openings 303 to allow more opportunities for the correct agglomerate sizes to enter the system. Sometimes one or more holes will be clogged with oversized agglomerates. Having multiple openings 303 allows harvesting to continue even during this condition.
2. The width of a harvesting cannula 100 opening 303, where the width of one or more harvesting cannula 100 openings 303 on a side is defined as the smallest dimension of one or more harvesting cannula 100 openings 303 on a side, will not exceed the inner diameter of the harvesting cannula 100. This will prevent the possibility of allowing agglomerate sizes through a harvesting cannula 100 opening 303 that won't fit through the inner lumen of the cannula 100.

Persons of ordinary skill in the art may appreciate that numerous design configurations may be possible to enjoy the functional benefits of the inventive kit. Thus, given the wide variety of configurations and arrangements of embodiments of the present invention, the scope of the invention is reflected by the breadth of the claims below rather than narrowed by the embodiments described above.

What is claimed is:

1. A “Process and System” whereby a kit of matching harvesting and reinjection cannulas for adipose tissue removal and reinjection procedures with intentional matching and compatible features, where the preferred embodiment includes features such that the area and size of the openings match, are used and intended to maximize tissue viability and minimize procedure time when used in the removal and replacement of adipose tissue, said set comprising the following:
a. at least one harvesting cannula (100) having a continuous, hollow tube provided with one or more harvesting opening(s) at or near the distal end for drawing in adipose tissue into the inner lumen of said hollow tube.
b. at least one reinjection cannula (100) having a continuous, hollow tube provided with one or more reinjection opening(s) at or near the distal end where adipose tissue will exit the inner lumen of said hollow tube.
c. the area of the reinjection cannula (100) opening(s) is larger than, equal to, or no smaller than 80% of the area of the harvesting cannula (100) opening(s) (303).

2. A set of cannulas of claim 1 wherein the width of the reinjection cannula (100) opening(s) are/is larger than, equal to, or no smaller than 80% of the width of the harvesting cannula (100) opening(s), where the width is defined as the smallest dimension when calculating the area of an oval or rectangular opening using length and width.

3. A set of cannulas of claim 1 wherein the inner cross sectional area (302) of the reinjection cannula (100) is larger than, equal to, or no smaller than 30% of the area of the harvesting cannula (100) opening(s).

4. A set of cannulas of claim 1 wherein:
a. the width of the reinjection cannula (100) opening(s) are/is larger than, equal to, or no smaller than 80% of the width of the harvesting cannula (100) opening(s), where the width is defined as the smallest dimension when calculating the area of an oval or rectangular opening using length and width.
b. the inner cross sectional area (302) of the reinjection cannula (100) is larger than, equal to, or no smaller than 30% of the area of the harvesting cannula (100) opening(s).

5. A “Process and System” whereby a kit of matching harvesting and reinjection cannulas for adipose tissue removal and reinjection procedures with intentional matching and compatible features, where the preferred embodiment includes features such that the area and size of the openings match, are used and intended to maximize tissue viability and minimize procedure time when used in the removal and replacement of adipose tissue, said set comprising the following:
a. at least one harvesting cannula (100) having a continuous, hollow tube provided with one or more harvesting opening(s) at or near the distal end for drawing in adipose tissue into the inner lumen of said hollow tube.
b. at least one reinjection cannula (100) having a continuous, hollow tube provided with one or more reinjection opening(s) at or near the distal end where adipose tissue will exit the inner lumen of said hollow tube.
c. the width of the reinjection cannula (100) opening(s) are/is larger than, equal to, or no smaller than 80% of the width of the harvesting cannula (100) opening(s), where the width is defined as the smallest dimension when calculating the area of an oval or rectangular opening using length and width.

6. A set of cannulas of claim 5 wherein the inner cross sectional area (302) of the reinjection cannula (100) is larger than, equal to, or no smaller than 30% of the area of the harvesting cannula (100) opening(s).

7. A “Process and System” whereby a kit of matching harvesting and reinjection cannulas for adipose tissue removal and reinjection procedures with intentional matching and compatible features, where the preferred embodiment includes features such that the area and size of the openings match, are used and intended to maximize tissue viability and minimize procedure time when used in the removal and replacement of adipose tissue, said set comprising the following:
a. at least one harvesting cannula (100) having a continuous, hollow tube provided with one or more harvesting opening(s) at or near the distal end for drawing in adipose tissue into the inner lumen of said hollow tube.
b. at least one reinjection cannula (100) having a continuous, hollow tube provided with one or more reinjection opening(s) at or near the distal end where adipose tissue will exit the inner lumen of said hollow tube.
c. the inner cross sectional area (302) of the reinjection cannula (100) is larger than, equal to, or no smaller than 30% of the area of the harvesting cannula (100) opening(s).
